(19) United States
(12) Patent Application Publication
(10) Pub. No.: US 2005/0260717 A1
(43) Pub. Date: Nov. 24, 2005

(54) Double transdominant fusion gene and protein

(76) Inventors: C. Estuardo Aguilar-Cordova, Houston, TX (US); John W. Belmont, Houston, TX (US); J. Wade Harper, Sugar Land, TX (US); Andrew Rice, Houston, TX (US); Janet S. Butel, Houston, TX (US); Javier Chinen, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/648,954

(22) Filed: Aug. 27, 2003

Related U.S. Application Data
(63) Continuation of application No. 08/399,264, filed on Mar. 6, 1995, now abandoned.

(51) Int. Cl. C12Q 1/68; C12P 21/06; C12P 19/34; C12N 5/00; C12N 5/02

(52) U.S. Cl. 435/91.33; 435/6; 435/69.1; 435/325

(57) ABSTRACT

A double transdominant fusion gene (trev) to simultaneously inhibit two functions essential for HIV expression was constructed by linking tat and rev transdominant mutants. Trev was shown to independently inhibit both Tat and Rev functions in a human T cell line. Stably expressed Trev localized within the nucleus and exhibited a greater combinatorial effect than either single transdominant gene alone. Cells transfected with Trev showed a stable 20 to 30 fold inhibition of HIV propagation and were protected against viral cytopathic effects. Simultaneous inhibition of two essential viral genes present significant advantages for potential gene therapy treatment of HIV infection.

### FIELD OF THE INVENTION

The present invention relates generally to a double transdominant fusion gene (trev) and a method of treating HIV infection. More particularly, it relates to a combination of a mutant tat and mutant rev genes to form a transdominant fusion gene.

### BACKGROUND OF INVENTION

“Intracellular immunization” is the concept that the introduction of an exogenous gene into cells will render such cells resistant to specific pathogens. Various strategies for intercellular immunization against HIV have been proposed, including antisense transcripts, ribozymes, suicide gene expression, RNA decoy expression and transdominant suppressors.

Tat and Rev are HIV-encoded regulatory proteins essential for efficient viral replication. Tat is a potent positive regulator which interacts with a stem loop RNA structure (TAR) located at the 5′ end of all HIV-1 transcripts. Tat is a small nuclear protein from which the first 67 amino acids are sufficient for transactivation. A conserved cysteine-rich region between amino acids 27 and 57 is important for protein-protein interactions and a basic region between amino acids 48 and 57 is required for nuclear localization and binding to TAR. Mutations in the latter have yielded potent transdominant suppressors. Rev is a 116-amino acid protein required for nuclear export of incompletely spliced transcripts necessary for HIV structural gene and full genomic expression. Rev contains an arginine-rich base dominant between amino acids 35 and 51 required for nucleolar localization and binding to the rev responsive element (RRE), and a leucine-rich dominant between amino acids 75 and 83 important for protein-protein interactions. Mutations in the protein-protein interaction domain have yielded effective transdominant suppressors of wild-type Rev function.

Liu et al., reported the addition of a TAR decoy to a Rev transdominant vector significantly enhanced the inhibitory effect of the transdominant construct. Liem et al., reported the construction of a retroviral vector which could express either a transdominant tat or a transdominant rev, this vector was significantly more effective than comparable vectors which expressed one transdominant and one wild-type tat or rev. Both of these reports suggest possible benefits of inhibiting tat and rev.

Most proposed strategies attack a single stage of viral expression. The present invention describes a fusion protein which simultaneously inhibits both proteins. It includes a double transdominant molecule which simultaneously inhibits tat and rev, two essential viral proteins. The double transdominant has functional advantages over single inhibitors.

### SUMMARY OF THE INVENTION

An object of the present invention is the provision of a double transdominant fusion gene.

An additional object of the present invention is the provision of a method for the treatment of HIV disease.

A further object of the present invention is the provision of a double transdominant fusion protein.

Thus, in accomplishing the foregoing objects there is provided in accordance with one aspect of the present invention, a double transdominant fusion gene, comprising: a tat transdominant mutant gene linked to a rev transdominant mutant gene, wherein said double transdominant fusion gene inhibits expression of HIV.

Its specific embodiments of the present invention codons in the tat transdominant mutant gene which code for basic amino acids at positions 52 to 57 of the Tat protein are replaced with codons which code for neutral amino acids.

More specifically, the codon sequences for the amino acids arg, arg, gln, arg, arg and arg are replaced with the codon sequences for the amino acids gly, gly, ala, gly, gly and gly.

In specific embodiments codons, in the rev transdominant mutant gene which code for amino acids at positions 80 to 82 of the Rev protein have been deleted.

In a preferred embodiment, the tat and rev transdominant mutant genes are linked by a histidine bridge.

An additional embodiment of the present invention includes the protein which is encoded by the double transdominant fusion gene.

Another embodiment of the present invention includes a methods of treating HIV disease in humans comprising delivering to the human to be treated a pharmacologically effective dose of a double transdominant gene containing a tat transdominant mutant gene linked to a rev transdominant mutant gene.

Other and further objects, features and advantages will be apparent and the invention will be more readily understood from a reading of the following specification and by reference to the accompanying drawings, forming a part thereof, where examples of the present preferred embodiments of the invention are given for purpose of disclosure.

### DESCRIPTION OF THE DRAWINGS

FIG. 1 is a schematic illustration of the structure of a double trev transdominant fusion gene and its double transdominant fusion protein.

FIG. 2 shows the comparison of inhibition of function by each single transdominant mutant genes (tat and rev) and the double transdominant gene (trev).

FIG. 3 shows the effects of transdominant constructs on transient expression of proviral vectors.

FIG. 4 shows that stable expression of Trev in 1G5 parental cells.

FIG. 5 shows a stable expression of Trev in 1G5-Trev cells.

FIG. 6 shows a comparison of the effects of stable transdominant genes (tat, rev, trev) on HIV-1 challenge.

FIG. 7 shows the results from a long-term cytopathicity protection assay.

FIG. 8 shows the p24 results of HIV-1_{NL4-3} infection of 1G5 cells transduced with the S3 Trev retroviral vector.

FIG. 9 shows the viable cell number results of HIV-1_{NL4-3} infection of 1G5 cells transduced with the S3 Trev retroviral vector.

FIG. 10 shows the effects of transdominant constructs on transient expression of a proviral vector.

FIG. 11 shows the effects of infection with HIV-1_{NL4-3} of stable transdominant cell lines.

FIG. 12 shows the effects of long-term HIV mediate cylopathicity in 1G5 and 1G5-Trev cells.

The drawings and figures are not necessarily to scale and certain features of the invention may be exaggerated in scale as shown in schematic form in the interest of clarity and conciseness.

### DETAILED DESCRIPTION OF INVENTION

It is readily apparent to one skilled in the art that various substitutions and modifications may be made to the invention disclosed herein without departing from the scope and spirit of the invention.

As used herein, the term “vector” refers to the means by which the transdominant mutant trev gene can be introduced into a host organism or a tissue. There are various types of vectors, including plasmids, bacteriophages and cosmids.

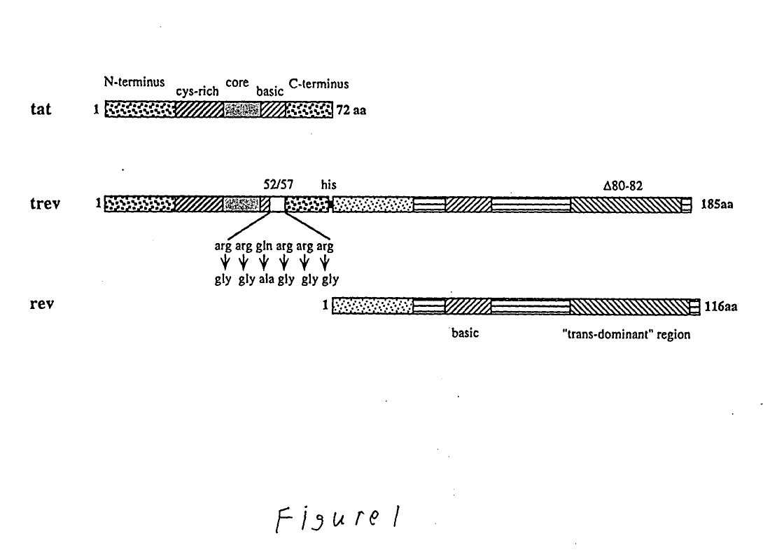

As used herein, the term “Tat” refers to a HIV coded regulatory protein essential for efficient viral replication. Tat is a potent positive regulator which acts on a stem loop RNA structure (TAR) located at the 5′ end of all HIV-1 transcripts. Tat is a small 86 amino acid nuclear protein from which the first 67 amino acids are sufficient for transactivation. A conserved cysteine-rich region between amino acids 27 to 37 is important for protein-protein interactions. A basic region between amino acids 48 to 57 is required for nuclear localization and binding to TAR. A schematic representation of Tat is shown in FIG. 1.

As used herein, the term “tat” refers to the gene which encodes Tat. A schematic representation of tat is shown in FIG. 1.

As used herein, the term Rev refers to a HIV coded regulatory protein essential for efficient viral replication. Rev is a 116-amino acid protein required for nuclear export of incompletely spliced transcripts necessary for HIV structural gene and full genome expression. Rev contains an arginine-rich basic domain between amino acids 35 to 51, which is required for nucleolar localization and binding to the rev responsive element (RRE), and a leucine rich domain between amino acids 75 to 83 which is important for protein-protein interactions. A schematic representation of Rev is shown in FIG. 1.

As used herein the term “rev” refers to the gene which encodes Rev. A schematic representation of rev is shown in FIG. 1.

As used herein, the term “Trev” refers to a protein containing transdominant portions of both a Tat and Rev, which portions have noncompeting modes of action. In Trev, the basic amino acids at positions 52 to 57 of the Tat portion have been replaced with neutral codons and the Rev portion contains a three-amino acid deletion at positions 80 to 82. The Tat and rev portions of Trev are connected by a histidine bridge. A schematic representation of Trev is shown in FIG. 1.

As used herein, the term “trev” refers to a double transdominant mutant gene comprised of a combination of at least one tat and at least one rev transdominant mutant gene. A schematic representation of trev is shown in FIG. 1.

One embodiment of the present invention is a double transdominant fusion gene comprising a tat transdominant mutant gene linked to a rev transdominant mutant gene. The fusion gene, trev, is designed to express complete Tat and Rev transdominant proteins with non-competing modes of action. The double transdominant fusion gene will inhibit the expression of HIV.

In the preferred embodiment, the tat transdominant mutant gene has the codons which code for the basic amino acids at position 52 to 57 of the Tat protein replaced with the codons which code for neutral amino acids. One skilled in the art readily recognizes that a variety of substitutions are available.

In the preferred embodiment of the present invention, the codons for the Tat amino acid sequence arg, arg, gln, arg, arg and arg have been replaced with the codon sequence for the amino acids gly, gly, ala, gly, gly and gly.

In the preferred embodiment of the present invention, the rev transdominant mutant gene has the codons which code for amino acids at positions 80 to 82 of the rev protein deleted.

In the preferred invention, the tat and rev transdominant mutant genes are linked by a histidine bridge.

Another embodiment of the present invention includes a method for treating HIV disease in humans comprising delivering to the human being treated a pharmacological dose of a double transdominant gene containing a tat transdominant mutant gene linked to a rev transdominant mutant gene.

The composition of the present invention can be formulated according to known methods to prepare pharmacologically useful compositions. The compositions of the present invention or their functional derivatives are combined in admixture with a pharmacologically acceptable carrier vehicle. Suitable vehicles and their formulations are well known in the art. In order to form a pharmacologically acceptable composition suitable for effective administration, such compositions will contain an effective amount of the double transdominant fusion gene or its equivalent or the functional derivative thereof, together with the suitable amount of carrier vehicle.

The composition of the present invention will usually be formulated in a vector. The vector can be administered by a variety of methods including parenterally, by injection, rapid infusion, nasopharyngeal absorption, dermal absorption or orally. The compositions may alternatively be administered intramuscularly or intravenously. In addition the vector for parenteral administration can further include sterile aqueous or nonaqueous solutions, suspensions and emulsions. Examples of known nonaqueous solvents include propylene glycol, polyethylene glycol, vegetable oils, such as olive oil, and injectable organic esters, such as ethyl oleate. Carriers, adjuncts or occlusive dressings can be used to increase tissue permeability and enhance absorption. Liquid dosage forms for oral administration may generally comprise a liposome solution. Suitable forms for suspension include emulsions, solutions, syrups and elixirs containing inert diluents commonly used in the art, such as purified water. Besides the inert dilutants, such compositions can also include wetting agents, emulsifying and suspending agents or sweetening, flavoring, coloring or perfuming agents.

Additionally, pharmaceutical methods may be employed to control the duration of action. These are well known in the art and include control release preparations and can include appropriate macromolecules, for example polymers, polyesters, polyamino acids, polyvinyl, pyrolidone, ethylenevinylactate, methylcellulose, caroboxymethylcellulose, or protamine sulfate. The concentration of macromolecules, as well as the methods of incorporation, can be adjusted in order to control release. Additionally, the vector could be incorporated into particles of polymeric materials such as polyesters, polyamino acids, hydrogells, poly (lactic acid) or ethylene vinylacetate co-polymers. In addition to being incorporated, these agents can also be used to trap the vectors in microcapsules. These techniques are well known in the art.

A composition is said to be “pharmacologically acceptable” if its administration can be tolerated by a recipient patient. Such an agent is said to be administered in a “therapeutically effective amount” if the amount administered is physiologically significant. An agent is physiologically significant if its presence results in detectable change in the physiology of a recipient patient.

Generally, the dosage needed to provide an effective amount of composition will vary depending on such factors as the recipient's age, condition, sex and extent of disease, if any, and other variables which can be adjusted by one of ordinary skill in the art.

The following examples are offered by way of illustration and are not intended to limit the invention in any manner.

### EXAMPLE 1

### Plasmid Construction

The fusion gene, trev, was designed to express complete Tat and Rev transdominant proteins with noncompeting modes of action (FIG. 1). Generally, in trev the basic amino acids at positions 52 to 57 of Tat were substituted for neutral codons (tat 52/57) and joined by a histidine bridge to a three amino acid deletion at positions 80 to 82 of Rev (revΔ80-82). Both sequence changes have independently been shown to generate potent negative single transdominants.

The mode of action of Tat 52/57 is not known but does not involve competition for TAR binding nor inhibition of wild type Tat from nuclear localization and is thought to function by competition for cellular factors. RevΔ80-82 localizes to the nucleus and is thought to function by binding to RRE but not to necessary cellular factors. Thus, in Trev the Tat portion may be competing with wild type Tat for soluble cellular factors while the Rev portion is competing with wild type Rev for RRE binding. These two modes of action should not interfere with each other. A fusion construct was chosen to assure simultaneous expression of both functions and allow greater versatility of choices for delivery systems.

The specific construction of trev is depicted in FIG. 1. The first 72 amino acids from HIV 1-Tat with the indicated substitutions were amplified by PCR from the Tat transdominant gene, tat 52 - 57 using 5′-CGCGCATATGGCAGGAAGAAGCGGAG-3′ as a 5′ primer and 5′-CTAACAGATCTATTCTTTAGCTCCTGACTCCAA-3′ as a 3′ primer. The amplification product was cloned into the HincII site of pBluescript KS (Stratagene, La Jolla, Calif., USA) and sequenced to verify the presence of the desired substitutions. An NdeI site was created at the 3′ end of tat 52 - 57 for in-frame insertion of rev. The 80-82 deletion in the Rev coding sequence was obtained as an NdeI-EcoRI fragment in pBR322 and subcloned into NdeI-EcoRI of the above construct. The final product was sequenced to verify transdominant mutations and in-frame open reading sequences for both tat and rev portions. An RsaI-SpeI fragment containing the trev sequence was substituted for Bgal into the SmaI-XbaI site of pPGK-Bgal. The PGK (phosphoglycerate kinase) promoter has been shown to provide efficient expression in the hematopoietic cell lineage. The Tat 52 - 57 expression vector was pDex (RSV-LTR, tat 52/57, SV-40 poly A site). The Rev M10 expression vector was pBC12-M10 [cytomegalovirus (CMV)-immediate-early promoter, Rev-M10, rat pre-pro insulin intron-poly A site].

pRLR was constructed by cloning a 2.2-kb fragment containing an RSV-LTR promoter and the firefly luciferase coding sequence without a ploy-adenylation signal 5′ to a 4.4-kb fragment of HIV-1 containing most of the env sequence, the RRE and the 3′ LTR and poly A signal in a pBluescript backbone.

pS3Trev was constructed by subcloning a 1450-bp SalI to NotI PGK-trev fragment from pPGK-trev into the SalI and StuI sites in the body of the pS3 retroviral vector backbone.

### EXAMPLE 2

### Cell Culture and Analysis

Cells were maintained in RPMI 1649 (GIBCO BRL, Gaitherburg, Md. USA) with 10% calf serum (HyClone, Logan, Utah, USA) at densities of less than 10⁶ cells per millimeter. All transfections were carried out in triplicate by electroporation in 125 μl of growth medium, 125 V, 3000 μF in a BTX-ECM600 electroporator. The total amount of DNA per transfection was adjusted to 20 μg using pUC-12 as carrier. Unless otherwise indicated, the concentrations were 1 μg per transfection of effector plasmid (wild-type tat or HIV-1) and 5 μg per transfection of the transdominant gene plasmid. Luciferase analyses were performed 16-24 h after transfection using the Luciferase Analysis System (Promega, Madison Wis., USA), as per the recommendation of the manufacturer. Cell viability was measured by trypan blue exclusion and p24 antigen was measured by ELISA from 50 μl of cell supernatant (Coulter).

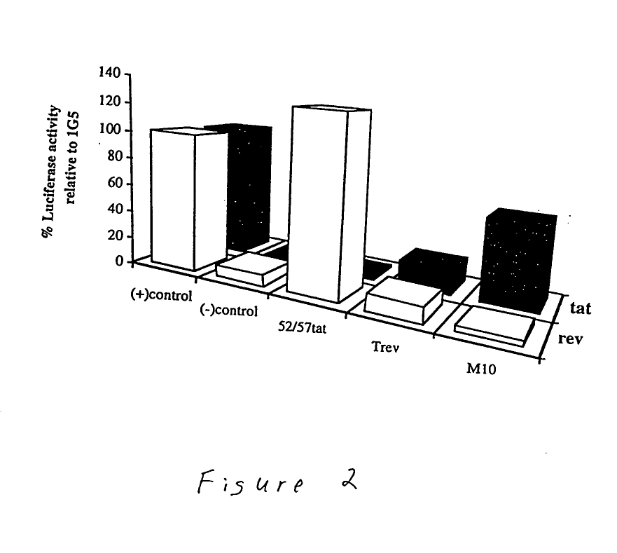

In FIGS. 2, 3 and 10, the Tat analyses were done by cotransfection of wild type Tat with the transdominant plasmid in 1:5 molar ratio into the reporter cell line 1G5. Positive control shows the effects of wild type Tat without a transdominant, negative control shows the effect of carrier DNA alone. Rev transdominant analyses were done by cotransfection of pRLR (1 μg) with wild type Rev (1 μg) and the transdominant plasmid in a 1:5 molar ratio into Jurkat cells. In FIG. 10, the proviral clone HIV-1_{NL4-3} was cotransfected into 1G5 reporter cells.

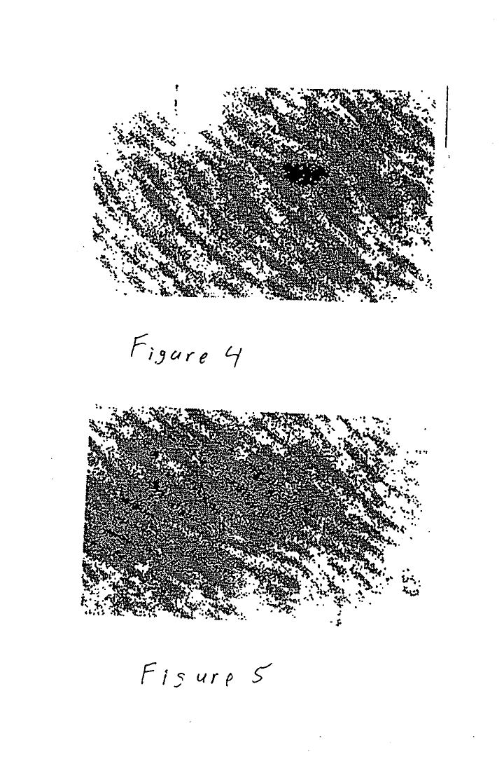

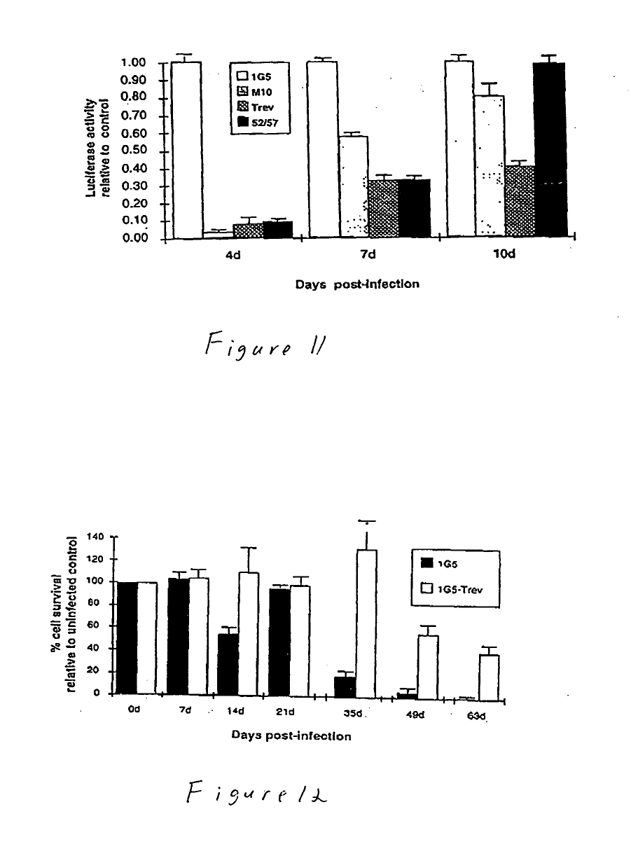

In FIGS. 6 and 11, triplicates of 2.5×10⁵ parental (1G5) and stably transfected cells were seeded in six well plates. The cells were all infected with equal aliquots of a low titer HIV-1_{NL4-3} virus stock on day 1. Every three days 2.5×10⁵ cells were harvested from each well for luciferase activity analysis and 2.5×10⁵ cells were re-seeded in fresh media for subsequent analysis.

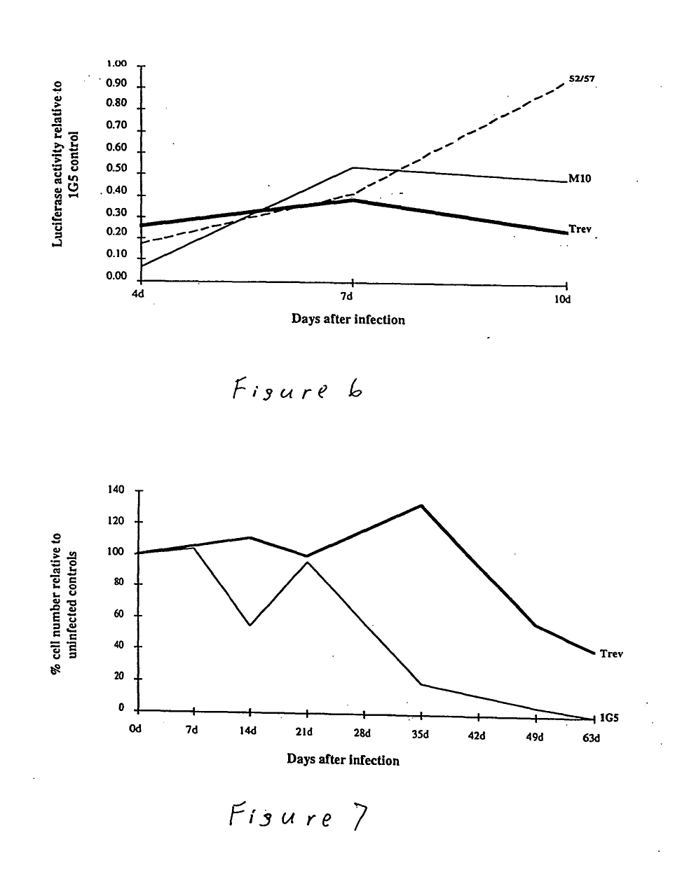

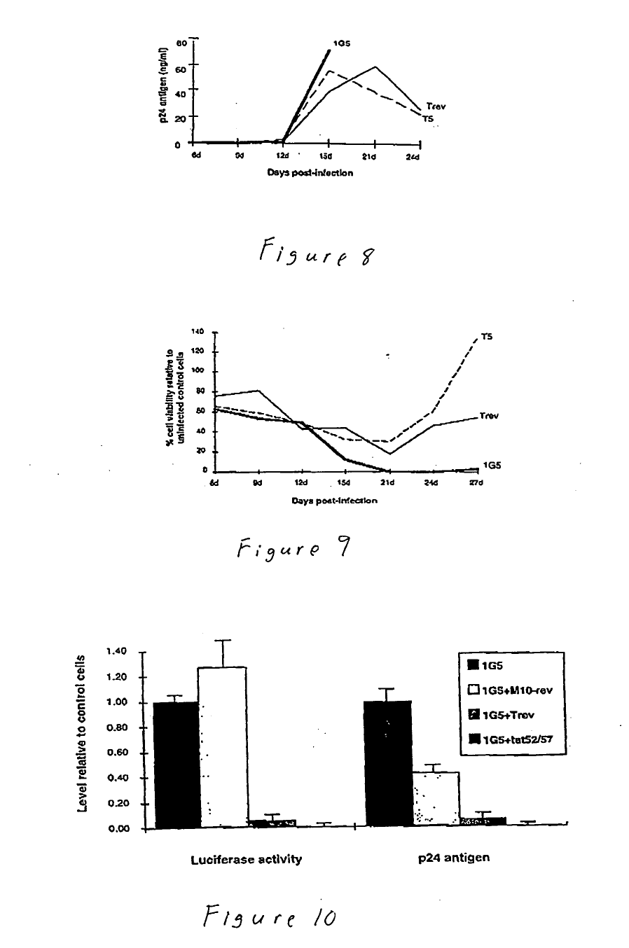

In FIGS. 7 and 12, two six-well dishes each with triplicates of 2.5×10⁶ parental (1G5) and 1G5-Trev cells were seeded at day 0. One set of triplicates was infected with equal aliquots of a low titer HIV-1_{NL4-3} virus stock and the second set was used as control. Every seven days (14 days for last data point) the cultures were split 1:4. 75% of the cells were removed and counted by Trypan Blue exclusion, 25% were used to maintain the culture.

HIV infection was done by direct addition of viral supernatant to the growth medium at an approximate multiplicity of infection of 0.001. Tat retroviral vector transduction was done by exposure of the cells to the viral supernatant in growth medium supplemented with Polybrene to 4 μg/ml. After overnight incubation the cells were changed to normal growth medium.

### EXAMPLE 3

### Immunocytochemistry

Cytospin preparations were fixed with cold methanol for 3 min, blocked with a 10% solution of normal goal serum and incubated with a 1:800 dilution of rabbit anti-Rev primary antibody (Art 27/51) overnight at 40° C.²⁰. Detection was accomplished using the suggested super-sensitive conditions from a StrAviGen MultiLink kit (Biogenex, San Ramon, Calif., USA) followed by stable DAB from Research Genetics (Huntsville, Ala., USA) at r.t. for 3 min and a hematoxylin counterstain.

### EXAMPLE 4

### Cotransfection

Trev was evaluated for lack of Tat or Rev transactivating activity and independent Tat and Rev transdominant activity. In cotransfection experiments with Tat dependent and Rev dependent luciferase constructs, Trev showed no transactivation potential.

A. Tat Transdominant Activity. Tat transdominant activity was evaluated by cotransfection with wild type Tat into the reporter cell line 1G5. These cells contain an integrated HIV long terminal repeat (HIV^{LTR}) luciferase construct highly sensitive to Tat transactivation. Trev showed approximately an eight fold inhibition of Tat transactivation compared to about 30 fold observed with the single transdominant Tat 52/57 (FIG. 2). Comparable inhibition results were obtained with a transient Tat-dependent HIV^{LTR} luciferase construct in cells derived from the human Jurkat T-cell line. Trev was also effective on a 1G5 clone with stable constitutive Tat expression.

B. Rev Transdominant Activity. Rev transdominant activity was analyzed by cotransfection of a Rev dependent luciferase construct with wild type Rev and Trev into Jurkat cells (pRLR). Trev inhibited Rev transactivation by up to 10 fold, comparable to the inhibition observed with an equivalent single transdominant, M10 (FIG. 2). The mechanism for the observed near two fold inhibition by M10 in the Tat assay is not evident, but probably reflects nonspecific plasmid effects. However, M10 was not inhibitory, but rather stimulated Tat effects when a full proviral HIV construct was used. Only the fusion protein Trev was efficient in independently inhibiting both Tat and Rev function.

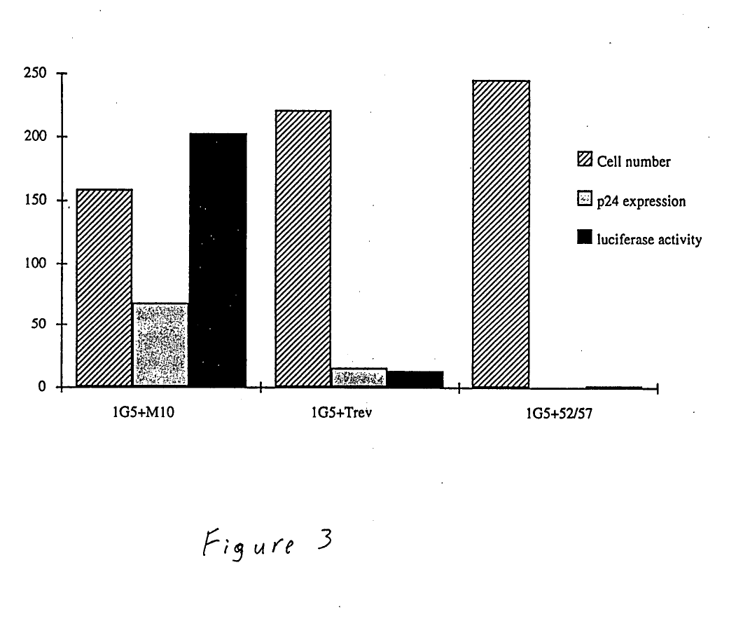

C. Simultaneous Tat and Rev Activity. Simultaneous inhibition of Tat and Rev was analyzed by cotransfection with a provirus clone of HIV-1_{NL4-3} and infection of stably transfected cells with HIV-1_{NL4-3} virions. Cell viability was measured to evaluate protection against cytopathic effects, luciferase was analyzed as an indicator of Tat activity and p24 was used as an indicator of viral particle production. The single transdominant Tat 52/57 was most efficient in transient transfection assays, closely followed by Trev (see FIGS. 3 and 10). The single Rev transdominant M10, however, showed a surprising two fold increase in luciferase activity; although it did protect against cytopathic effects and viral particle production. The increase in luciferase activity promoted by M10 may reflect an increased production of multiply spliced HIV transcripts (therefore Tat) as a result of the inability to export incompletely spliced transcripts from the nucleus. This would create a potent positive feedback loop.

To further analyze effects of coordinate Tat and Rev inhibition the double and single transdominants were stably expressed in 1G5 cells. Populations of transfected cells were studied to minimize possible effects of clonal variation on transdominant gene expression or susceptibility to HIV infection. Expression of transdominants in the stable lines was analyzed by transient inhibition assays of Tat transactivation in Tat 52/57 and Trev transfected cells and of Rev in M10 and Trev transfected cells. All stable cell lines retained their relative transdominant activities and there were no detectable deleterious effects on the growth characteristics of the transfected cell populations. Tat 52/57 localized to both the cytoplasm and nucleus and RevΔ80-82 was shown to localize to the nucleus. In the stably transfected cells, Trev protein was shown to localize to the nucleus by immunocytochemistry (FIGS. 4 and 5), indicating that Trev retained a structure for appropriate subcellular localization.

Stable transfected cell populations were then challenged with infectious HIV-1_{NL4-3}. All three transdominant genes showed significant suppressor effects four days after HIV infection. However, suppression by Tat 52/57 was short in duration and approached control levels between seven and 10 days after infection (FIGS. 6 and 11). Inhibition by M10 reached a plateau at approximately 50% inhibition for up to ten days. Trev transfected cells showed a 75% inhibition for the same length of time (FIGS. 6 and 11).

To confirm the inhibition observed was not a result of undetected down-regulation of CD4 expression, 1G5 parental and 1G5-Trev cells were transduced with an amphotropic murine leukemia virus (MuLV) based Tat retroviral vector which does not require CD4 for infection. 1G5-Trev cells showed a 50-60% inhibition compared to the parental cell line. 1G5-Trev cells were also compared with 1G5 parental cells on a long term cell survival and luciferase activity assay. Trev showed continuous long term suppression of luciferase activity and a significant reduction of the cytopathic effects of HIV infection for the 63 day duration of the experiment (FIGS. 7 and 12).

### EXAMPLE 5

### Gene Therapy

HIV-1 gene therapy will likely require stable transduction of pluripotent hematopoietic progenitor cells and retroviral vectors are the vehicle of choice for stable gene transduction of these cells, therefore, Trev was subcloned into the S3 backbone and virions produced (S3 Trev) in the GP+AM12 packaging cells. Trev sequences were detected in S3 Trev-transduced 1G5 cells by polymerase chain reaction (PCR) analysis. An S3 Trev-transduced population was challenged with HIV_{NL4-3} and showed increased resistance to HIV_{NL4-3} cytopathic effects for up to 27 days (FIGS. 8 and 9).

Use of transdominant suppressors as effectors in gene therapy strategies for HIV infection have been proposed for Tat, Rev and Gag proteins. The importance of Tat and Rev in the viral life cycle and their distinctiveness from cellular genes make them attractive targets for therapeutic intervention. Transdominant Tat proteins very efficiently inhibit acute Tat mediated transactivation. However, Tat independent HIV transcription can still occur and long term stable effects of Tat transdominants have not been achieved. Therefore, inhibition of Tat function alone is not likely to be sufficient as an antiviral treatment. Inhibition of Rev function decreases viral structural gene production but may indirectly lead to increased production of regulatory proteins, including Tat and Rev itself. Increased Tat expression may further augment HIV transcription and Tat and Rev production, eventually overcoming Rev transdominant function and allowing production of virus or causing cellular dysfunction as a direct result of Tat or Rev expression. Inhibition of both regulatory proteins simultaneously may prevent this self-defeating feedback loop and thus complement the effect of a single transdominant. The double transdominant effects of Trev are functionally distinct and were shown to be superior to the counterpart single transdominants in stable protection against HIV infection. Trev represents a potent novel molecule for gene therapy treatment of HIV infection.

One skilled in the art will readily appreciate that the present invention is well adapted to carry out the objects and obtain the ends and advantage mentioned, as well as those inherent therein. The nucleotides, proteins, peptides, methods, procedures and techniques described herein are presently representative of the preferred embodiments, are intended to be exemplary and are not intended as limitations on the scope. Changes therein and other uses will occur to those skilled in the art which are encompassed within the spirit of the invention or defined by the scope of attached claims.

1. (canceled)

2. (canceled)

3. (canceled)

4. (canceled)

5. (canceled)

6. An isolated double transdominant fusion gene, comprising:
a tat transdominant mutant gene, wherein codons of said tat mutant gene which code for basic amino acids at positions 52 to 57 of the Tat protein are replaced with codons which code for neutral amino acids;
a rev transdominant mutant gene, wherein the codons of the rev mutant which code for amino acids at positions 80 to 82 of the Rev protein have been deleted; and
codon coding for histidine, wherein said histidine comprises a histidine bridge that links the Tat transdominant mutant protein to the Rev transdominant mutant protein.

7. (canceled)

8. (canceled)

9. A vector comprising said isolated double transdominant fusion gene of claim 6, wherein said gene is operably linked to a promoter.

10. A transdominant protein produced by the vector of claim 9.

11. An isolated double transdominant fusion gene, comprising a tat transdominant mutant gene having a substituted basic domain in which basic amino acids are replaced by neutral amino acids, said tat transdominant mutant gene is operably linked to a rev transdominant mutant gene having a deletion in a leucine-rich coding domain, wherein the double transdominant fusion gene product inhibits HIV expression.

12. The transdominant fusion gene of claim 11, wherein the tat and rev transdominant mutant genes are linked by a codon which codes for a histidine bridge.

13. A vector comprising said isolated double transdominant fusion gene of claim 11, wherein said gene is operably linked to a promoter.

14. A transdominant protein produced by the vector of claim 13.

15. A method of inhibiting HIV replication in a HIV infected cell in vitro, comprising:
delivering to the HIV infected cell in vitro an effective amount of the vector of claim 13, from which is expressed the double transdominant fusion gene product.


SEQUENCE LISTING (2 sequences)

SEQ ID NO: 1 - DNA (genomic), 26 bp; single-stranded, linear
  units: 26 bp
cgcgcatatggcaggaagaagcggag

SEQ ID NO: 2 - DNA (genomic), 33 bp; single-stranded, linear
  units: 33 bp
ctaacagatctattctttagctcctgactccaa